(19) United States
(12) Patent Application Publication
(10) Pub. No.: US 2006/0064770 A1
(43) Pub. Date: Mar. 23, 2006

(54) Methods for identifying a cell or an embryo carrying a Y chromosome

(76) Inventors: David Frendewey, New York, NY (US); Wojtek Auerbach, Ridgewood, NJ (US); Thomas M. Dechiara, Katonah, NY (US); David M. Valenzuela, Yorktown Heights, NY (US)

Correspondence Address: REGENERON PHARMACEUTICALS, INC, 777 OLD SAW MILL RIVER ROAD, TARRYTOWN, NY, 10591, US

(21) Appl. No.: 11/227,848

(22) Filed: Sep. 15, 2005

Related U.S. Application Data
(60) Provisional application No. 60/611,381, filed on Sep. 20, 2004.

(51) Int. Cl. A01K 67/027; C12N 5/06; C12N 15/87

(52) U.S. Cl. 800/18; 435/455; 435/354

(57) ABSTRACT

Methods for modifying a mammalian cell target such that the presence of a Y chromosome is detectable. The method includes construction of a targeting vector with a detectable marker that can recombine or insert into a pre-selected site on the Y chromosome, allowing the presence of the Y chromosome to be detectable through the presence of the integrated detectable marker. The method can be used to distinguish male from female embryos.

### CROSS-REFERENCE TO RELATED APPLICATIONS

This application claims the benefit under 35 USC § 119(e) of U.S. Provisional 60/611,381 filed 20 Sep. 2004, which applications are herein specifically incorporated by reference in its entirety.

### BACKGROUND

1. Field of the Invention

The invention is directed to methods for modifying a Y chromosome. More particularly, the present invention is directed to methods for modifying and/or tagging a Y chromosome for subsequent detection.

2. Description of the Related Art

Methods for modifying genes in eukaryotic cells are known in the art. See, for example, U.S. Pat. No. 6,586,251. Additionally, Rohozinski et al. describe methods for the insertional targeting of mouse Y chromosome genes based on 5′ hrpt phage vectors (Genesis (2003) 32:1-7).

### BRIEF SUMMARY OF THE INVENTION

In a first aspect, the invention features a method for generating a eukaryotic cell having a detectable Y chromosome, comprising in the 5′ to 3′ direction: (a) generating a Y chromosome targeting vector, wherein the targeting vector comprises a 5′ homology arm, a promoter, a reporter gene operably associated with the promoter, and a 3′ homology arm, wherein the 5′ and 3′ homology arms are homologous to a region of the Y chromosome; and (b) introducing the targeting vector of step (a) into a eukaryotic cell, such that a eukaryotic cell having a detectable Y chromosome is generated. The targeting vector can be introduced into eukaryotic cells by any method known to a one of skill in the art. These methods include, for example, transfection, electroporation and microinjection. In one embodiment, the targeting vector is introduced into eukaryotic cells by transfection.

Verification that a cell having a detectable Y chromosome has been generated may be accomplished in a number of ways known in the art. In one embodiment, a cell comprising a detectable Y chromosome is identified first by identifying cells comprising the reporter gene, e.g., when the reporter gene encodes a fluorescent protein, cells comprising the reporter gene may be isolated by FACS. The location of the reporter gene can then be verified by any method known to the art, including Southern blot analysis, PCR, FISH, etc. Preferably, verification of the integration site of the targeting vector into the Y chromosome is performed by a PCR method.

In one embodiment, the homology arms are homologous to a region of the mouse Y chromosome approximately 7 kb downstream from the 3′ UTR of the Sry gene (ENSEMBL accession number ENSMUSG00000043876, DNA sequence location AC140408.2.14022.19681). In a more specific embodiment, the homology arms are homologous to mouse Y chromosome regions approximately 3518141 to 3523030 (according to ENSEMBL nomenclature). In an even more specific embodiment, the homology arms are homologous to a mouse Y chromosome region at about 3520465, which region includes a NheI restriction site (located at DNA sequence AC140408.2.31830.42342). This region of the Y chromosome does not appear to interfere with the normal function of Y chromosome genes.

The targeting vector may be an insertion vector or a replacement vector. Examples of each are shown in FIGS. 1-4.

In one embodiment, the eukaryotic cell is an embryonic stem (ES) cell. More preferably, the stem cell is a mouse ES cell.

In one embodiment, the reporter gene encodes a fluorescent protein, such as, for example, GFP, eGFP, YFP, eYFP, DsRed, etc. The reporter gene is operably associated with a eukaryotic promoter capable of driving expression of the reporter gene in a eukaryotic cell. A preferred eukaryotic promoter is that from the human ubiquitin C gene.

In one embodiment, the targeting vector further comprises at least one promoter, and a selectable marker gene operably associated with the promoter. In one embodiment, the selectable marker gene may be a drug resistance gene such as a neomycin phosphotransferase gene (neo^{r}), a hygromycin B phosphotransferase gene (hyg^{r}), a herpes simplex virus tyrosine kinase gene (HSV-tk), etc. In a specific embodiment, the selectable marker gene is operably associated with a prokaryotic promoter, such as, for example, EM7, which allows a plasmid containing the targeting vector to be amplified in prokaryotic cells. In another embodiment, the selectable marker is further operably associated with a eukaryotic promoter, such as those associated with the reporter gene. The prokaryotic and the eukaryotic promoters can be arranged sequentially. In a specific embodiment, the prokaryotic promoter is embedded in the eukaryotic promoter.

In one embodiment, the selectable marker gene and its operably associated promoter is flanked by a pair of site-specific recombinase recognition sites, e.g., loxP, Frt, or other recombinase sites known in the art.

In a second aspect, the invention features a method for generating a transgenic animal comprising a detectable Y chromosome, the method comprising: (a) generating a Y chromosome targeting vector, wherein the targeting vector comprises a 5′ homology arm, a promoter, a reporter gene operably associated with the promoter, and a 3′ homology arm, wherein the 5′ and 3′ homology arms are homologous to a region of the Y chromosome; (b) introducing the Y chromosome targeting vector of (a) into a eukaryotic embryonic stem cell; (c) introducing the cell of step (b) into an embryo; and (d) introducing the embryo into a surrogate mother for gestation.

In one embodiment, the embryo into which the modified ES cell is introduced is a pre-implantation embryo. Preferably, the embryo is a blastocyst stage embryo.

In a third aspect, the invention features a method for identifying a female embryo, comprising (a) generating a Y chromosome targeting vector, wherein the targeting vector comprises a 5′ homology arm, a promoter, a reporter gene operably associated with the eukaryotic promoter, and a 3′ homology arm, wherein the 5′ and 3′ homology arms are homologous to a region of the Y chromosome; (b) introducing the Y chromosome targeting vector into a eukaryotic embryonic stem (ES) cell; (c) introducing the cell of step (b) into an embryo; (d) introducing the embryo into a surrogate mother for gestation, wherein an animal is produced; (e) identifying a male animal produced in step (d) producing sperm having a detectable Y chromosome; (f) breeding the male animal of step (e) to a female animal such that embryos are generated; (g) harvesting the embryos generated in step (f); and (h) identifying an embryo lacking a marked Y chromosome, wherein such an embryo is female.

In one embodiment, the reporter gene encodes a fluorescent protein and the embryos with or without a marked Y chromosome are distinguished by visual inspection or fluorescence under the light of an appropriate exciting wavelength.

In a fourth aspect, the invention features a method for distinguishing between male and female embryos, comprising (a) generating a Y chromosome targeting vector, wherein the targeting vector comprises a 5′ homology arm, a promoter, a reporter gene operably associated with the eukaryotic promoter, and a 3′ homology arm, wherein the 5′ and 3′ homology arms are homologous to a region of the Y chromosome; (b) transfecting a eukaryotic embryonic stem (ES) cell with the Y chromosome targeting vector of (a); (c) introducing the cell of step (b) into an embryo; (d) introducing the embryo into a surrogate mother for gestation, wherein an animal is produced; (e) identifying a male animal produced in step (d) producing sperm having a detectable Y chromosome; (f) breeding the male animal of step (e) to a female animal such that embryos are generated; (g) harvesting the embryos generated in step (f); and (g) distinguishing embryos with a marked Y chromosome from embryos without a marked Y chromosome, wherein embryos lacking a marked Y chromosome are female embryos and embryos having a marked Y chromosome are male embryos.

In a fifth aspect, the invention features an embryo comprising a detectable Y chromosome, generated by the method of the invention. In a related sixth aspect, the invention features a transgenic male animal comprising a detectable Y chromosome generated by the method of the invention.

Other objects and advantages will become apparent from a review of the ensuing detailed description.

### BRIEF DESCRIPTION OF THE FIGURES

FIG. 1 is a schematic representation of an embodiment of an insertion vector of the invention. hUb1=human ubiquitin C promoter; eGFP=enhanced green fluorescent protein coding region and a polyadenylation (polyA) signal; black shaded box=Frt site-specific recombinase recognition site; EM7=prokaryotic promoter; homology box=nucleotide sequences homologous to nucleotide sequences contained within the Y chromosome target region; neo^{r}=neomycin phosphotransferase coding region and a polyadenylation (polyA) signal; Nhe I=cleavage site of the Nhe I restriction enzyme.

FIG. 2 is a schematic representation of an embodiment of an insertion vector of the invention. The abbreviations are as in FIG. 1.

FIG. 3 is a schematic representation of an embodiment of a replacement vector of the invention. The abbreviations are as in FIG. 1; HB1=homology box 1; HB2=homology box 2; linearization site=a restriction enzyme cleavage site for linearizing the vector.

FIG. 4 is a schematic representation of an embodiment of a replacement vector of the invention. The abbreviations are the same as above.

### DETAILED DESCRIPTION

Before the methods, constructs and transgenic animals of the present invention are described, it is to be understood that this invention is not limited to particular methods, constructs, transgenic animals, and experimental conditions described, as such all may vary. It is also to be understood that the terminology used herein is for the purpose of describing particular embodiments only, and is not intended to be limiting, since the scope of the present invention will be limited only by the appended claims.

As used in this specification and in the appended claims, the singular forms “a”, “an” and “the” include plural references unless the context clearly dictates otherwise, e.g., “a cell” includes a plurality of cells. Thus, for example, a reference to “a method” includes one or more methods, and/or steps of the type described herein and/or which will become apparent to those persons skilled in the art upon reading this disclosure.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, constructs and materials are now described. All publications mentioned herein are incorporated herein by reference in their entirety.

Definitions

By “ES cell” as used herein is meant an embryonic stem cell. An ES cell can be derived from the inner cell mass of a blastocyst-stage embryo. By “blastocyst” is meant the mammalian conceptus in the post-morula stage, comprising the trophoblast and the inner cell mass. An “ES cell clone” as used herein is a subpopulation of cells derived from a single ES cell following introduction of DNA and subsequent selection.

By “exogenous promoter” as used herein is meant a promoter that differs from the promoter(s) present in the targeted locus.

By “flanking DNA” as used herein is meant a segment of DNA that is contiguous with and adjacent to a particular point of reference. Similarly, “upstream” and “downstream” refer to flanking DNA sequences positioned 5′ and 3′, respectively, to a particular point of reference.

By “gene knockout” as used herein is meant a genetic modification resulting from the disruption of the genetic information encoded at a chromosomal locus. By “gene knockin” as used herein is meant a genetic modification resulting from the replacement or insertion of the genetic information encoded at a chromosomal locus with a different DNA sequence. By “knockout animal” is used herein is meant an animal in which a significant proportion of the animal's cells harbor a gene knockout. By “knockin animal” as used herein is meant an animal in which a significant proportion of the animal's cells harbor a genetic knockin.

By “gene targeting” as used herein is meant the modification of an endogenous chromosomal locus by the insertion into, deletion of, or replacement of the endogenous sequences via homologous recombination using a targeting vector.

By the term “marker,” “reporter,” or “tag” is generally meant a moiety that allows the detection of a molecule of interest, such as a protein expressed by a cell. In some embodiments, a selectable marker is used, e.g., a drug resistance gene[s] such as those that encode neomycin phosphotransferase (neo^{r}), hygromycin B phosphotransferase (hyg^{r}), herpes simplex virus tyrosine kinase (HSV-tk), etc. In other embodiments, a visually detectable marker is preferred, e.g., a fluorescent protein such as GFP, eGFP, YFP, eYFP, DsRed, etc. Preferably, in the present context, a reporter gene targeted to a Y chromosome locus allows detection of the presence of the Y chromosome, whereas a selectable marker is used to identify cells comprising the targeting vector, or relevant portion thereof.

By “recombinase” as used herein is meant an enzyme that recognizes specific nucleotide sequences termed “site-specific recombinase recognition sites” and that catalyzes rearrangement, e.g. deletion, inversion, insertion, exchange, of DNA segments between these sites. Recombinases can, for example, delete sequences between the same site-specific recombination sites on the same nucleic acid molecule if the sites are oriented in the same direction with respect to one another or invert the sequences between the same site-specific recombination sites on the same nucleic acid molecule if the sites are oriented in opposite directions with respect to one another.

By “targeting vector” as used herein is meant a DNA construct that comprises sequences “homologous” to endogenous chromosomal nucleic acid sequences flanking a desired genetic modification(s). The flanking homology sequences, referred to as “homology arms,” direct the targeting vector to a specific chromosomal location within the genome by virtue of the homology that exists between the homology arms and the corresponding endogenous sequence and introduce the desired genetic modification by a process referred to as “homologous recombination.” By “homologous” as used herein is meant two or more nucleic acid sequences that are either identical or similar enough that they are able to hybridize to each other to undergo intermolecular exchange. The targeting vector of the invention may be a replacement vector or an insertion vector.

By “replacement vector” as used herein is meant a targeting vector that is capable of undergoing a double reciprocal recombination with a chromosomal location that replaces the chromosomal DNA with all components of the vector that are flanked on both sides by homologous sequences. Any heterologous sequences not contained within the vector homologous sequences do not stably integrate into the chromosomal locus.

By “insertion vector” as used herein is meant a targeting vector that is capable of undergoing a single reciprocal recombination or a double reciprocal recombination with its homologous chromosomal target that inserts into a chromosomal locus all components of the vector that are flanked by homologous sequences without replacing the chromosomal DNA.

General Description

One of the desired components of a transgenic animal study is the generation of a genetically modified transgenic animal capable of transmitting the genetic modification to progeny, i.e., a transgenic animal comprising the genetic modification in its germline. Current methods of creating such a transmission-capable transgenic animal tend to be inefficient in terms of resources and time expenditures. For example, to generate a genetically modified transgenic animal capable of transmitting the genetic modification to progeny, a modified ES cell heterozygous for a desired genetic modification is injected into a recipient embryo, and the recipient embryo is implanted into a surrogate mother for gestation and birth of transgenic progeny. The resulting transgenic progeny are chimeric because some of the progeny's tissues are derived from the injected ES cells while other of the progeny's tissues are derived from the recipient embryo cells. Because of this chimerism, the injected ES cells comprising the genetic modification may or may not form germline tissues in the progeny and be transmittable. To determine whether a chimera is capable of transmitting the genetic modification, the chimera must be bred to a wild type animal for the desired genetic modification to establish whether the resulting progeny (F1 progeny) have the genetic modification. If any of the F1 progeny of the cross between the chimera and the wild type animal are positive for the desired genetic modification, it is established that the chimera is capable of transmitting the desired genetic modification and the desired genetic modification is present in the germline of the animal. Typically, coat color markers are used to aid in the process of identifying transgenic animals, as known in the art.

The current need to generate an F1 generation to determine if the chimera is capable of transmitting the genetic modification is inefficient and costly in terms of time and cost of breeding and maintaining F1 progeny. One method of improving the efficiency of the process for generating transgenic animals is to inject male (XY) modified ES cells into female (XX) embryos. Sex bias among ES cell chimeras in favor of males is commonly observed when male ES cells are used. Conversion of female embryos to fertile, phenotypic male animals occurs when the male ES cells colonize sufficient portions of the tissues that determine sex in the developing embryo. Fully sex-converted chimeras are expected to transmit the ES cell genotype. Because the ES cell genotype includes the genetic modification of interest, the transmission of only the ES cell genotype by the chimera ensures that the animal is capable of transmitting the genetic modification. Thus, all male animals created by injecting modified male ES cells into female embryos will be able to transmit only the genetic materials from ES cells. However, such a method requires the ability to distinguish between male and female recipient embryos.

The present invention is directed to methods for identifying the sex of embryos that reduce the amount of time and resources necessary to identify transgenic animals capable of transmitting a desired genetic modification. By the methods of the present invention, female embryos can be identified and isolated from male embryos and prepared to receive ES cells carrying a desired genetic modification. By selectively implanting modified male ES cells into only female embryos, the resulting male chimeric animal will be capable of transmitting the desired genetic modification of the ES cell.

In general, the methods of the invention comprise: (1) creating a transgenic male animal comprising a modified Y chromosome that allows the presence of the Y chromosome to be detected; (2) breeding the male animal of (1) to a female animal to produce embryos; (3) harvesting the embryos; and (4) identifying the sex of each embryo based on the presence or absence of the Y chromosome. Embryos comprising the detectable Y chromosome are identified as male, whereas embryos without a detectable Y chromosome are identified as female. Once the female embryos have been identified, they can be prepared for injection of ES cells carrying a desired genetic modification. The invention also comprises nucleic acid constructs for modifying a Y chromosome and transgenic animals comprising the nucleic acid constructs integrated into their genomes.

The modification of a Y chromosome can comprise any modification that enables detection of the Y chromosome. In a specific embodiment, the Y chromosome is tagged with an enhanced green fluorescent protein (eGFP) gene, the expression of which is readily observable by known techniques. Accordingly, the sex of an embryo created by breeding a female to a male having a Y chromosome tagged with the eGFP gene can be determined based on testing for the presence of eGFP. The modification of the Y chromosome can be achieved by targeting vectors that comprise a reporter gene and recombine into a Y chromosome locus. In a specific embodiment, the modification of the Y chromosome is mediated by an insertion vector and/or a replacement vector that comprises the eGFP gene under the control of an exogenous promoter and sequences homologous to a selected Y chromosome locus for directing the vector to recombine into the homologous locus.

In an additional embodiment, modification of the Y chromosome may be achieved with a non-targeting vector comprising a reporter gene. In this embodiment, integration is random, and cells into which the non-targeting vector are introduced are screened to identify cells having a Y chromosome modified by chance. Methods for identifying integration of the vector into the Y chromosome are known to the art, for example, by PCR.

Selection of Gene(s) and/or Locus(Loci)

A variety of approaches can be used for selecting a gene or locus of interest for genetic mutation and/or modification of a Y chromosome. Selection can be based on specific criteria such as detailed structural or functional, or it can be selected in the absence of such detailed information as potential genes or gene fragments become predicted through the various genome sequencing projects. It should be noted that it is not necessary to know the complete sequence and gene structure of a gene or locus of interest to apply the methods of the invention.

According to one aspect, a gene and/or locus of interest is chosen based on its location on a Y chromosome and/or the technical feasibility of recombining an exogenous gene or fragment thereof therein. Various known genes and/or loci are amenable for targeting according to the present invention. Such known genes and/or loci include, but are not limited to, the Dby gene (Rohozinski et al. (2002) Genesis 32:1-7,), the Eif2s3y gene (Id.), and the Sry gene (Capel (1998) Ann Rev Physiol. 60:497-523).

The methods of the present invention can be practiced with regard to any Y chromosome gene or locus for which appropriately sized homologous sequences can be created through standard techniques (e.g., PCR elongation of oligonucleotide primers), as long as disruption of the locus does not result in infertility or the inability to produce progeny. The homologous sequences are incorporated into a targeting vector capable of recombining at the target locus. In one specific embodiment, a region on the mouse Y chromosome approximately 7 kb downstream from the 3′ UTR of the Sry gene (ENSEMBL accession number ENSMUSG0000043876, DNA sequence location AC140408.2.14022.19681) is suitable. More specifically, mouse Y chromosome regions approximately 3518141 to 3523030 (according to ENSEMBL nomenclature) can be targeted by the vectors and methods of the present invention. Even more specifically, a mouse Y chromosome region at approximately 3520465, which region includes a NheI restriction site (located at DNA sequence AC140408.2.31830.42342), is the locus targeted by the methods of the invention. Insertion of a reporter gene at this Y chromosome locus does not appear to interfere with the fertility related functioning of Y chromosome genes, and is therefore a desirable integration site due to the need for the generation of viable and normal functioning transgenic animals.

Genetic Mutations/Modifications

Although the preferred embodiments of the present invention are directed to recombining a reporter gene into a suitable Y chromosome gene and/or locus for aiding in the determination of the sex of an embryo, it is to be appreciated that the present invention is not so limited, and that it is generally applicable to making a variety of genetic mutations and/or modifications to a Y chromosome. Such genetic mutations and/or modifications can be performed through a variety of techniques, especially those disclosed in U.S. Pat. No. 6,586,251, and Valenzuela et al. (2003) Nature Biotechnology 21(6):652-659.

Nucleic Acid Constructs

The techniques used to obtain the components of the targeting vectors and to construct the targeting vectors described herein are standard molecular biology techniques well known to the skilled artisan (see e.g., Sambrook et al. (2001) Molecular Cloning: A Laboratory Manual, Third Edition, Vols. 1-3). Any of the vector construction methods known to one skilled in the art may be used to construct the targeting vectors of the invention. One standard molecular biology technique useful in practicing in the methods of the invention, especially in connection with the creation of replacement vectors, is bacterial homologous recombination. Bacterial homologous recombination, also commonly referred to as “recombineering,” can be performed in a variety of systems (Yang et al. (1997) Nat Biotechnol, 15:859-65). One example of a system currently in use is ET cloning (Zhang et al. (1998) Nat Genet, 20:123-8) and variations of this technology (Yu et al. (2000) Proc Natl Acad Sci USA, 97:5978-83). All DNA sequencing can be done by standard techniques using an ABI 373A DNA sequencer and Taq Dideoxy Terminator Cycle Sequencing Kit (Applied Biosystems, Inc., Foster City, Calif.).

Example targeting vectors useful for practicing the methods of the present invention can comprise the following components: a reporter gene for tagging the Y chromosome, an exogenous promoter operably associated with the reporter gene, a selectable marker gene for facilitating the process of amplifying the vector, at least one promoter operably associated with the selectable marker gene, homologous sequences for directing the vector to recombine at a target locus, and a plasmid backbone. The targeting vectors of the present invention can be configured as insertion vectors and/or replacement vectors.

According to the methods of the present invention, the expression of the reporter allows the presence of the Y chromosome to be detected, resulting in identification of a desired embryo. Accordingly, the reporter gene preferably expresses a protein that is readily detectable. Especially useful reporter genes are genes that facilitate rapid and simple identification of their presence. According to one embodiment, the reporter genes encoding fluorescent proteins, such as cyano fluorescent protein (CFP), green fluorescent protein (GFP), enhanced GFP (eGFP), yellow fluorescent protein (YFP), enhanced YFP (eYFP), blue fluorescent protein (BFP), enhanced BFP (eBFP), red fluorescent protein from the Discosoma coral (DsRed), MmGFP (Zernicka-Goetz et al. (1997) Development 124:1133-1137) or others familiar to skilled artisans. According to a preferred embodiment, the reporter gene is the fluorescent reporter eGFP gene. To increase the signals that a reporter gene produces, the reporter gene (e.g., eGFP reporter gene) may be present in the vector in multiple copies, such as in a tandem array (FIG. 2). Integration of a tandem array of reporter genes into the target locus provides for enhanced levels of reporter gene transcription products, thereby enhancing the functionality of an assay for identifying the presence of the marker gene product. The multiple reporter gene construct may suitably be created through standard techniques as known to a skilled artisan. In one embodiment, multiple copies of one reporter gene each driven by its own promoter can be used. In a specific embodiment, the vector includes three copies of the marker gene, such as the eGFP gene, each operably associated with a promoter, such as the hUbC promoter. Alternatively, multiple copies of one reporter gene each separated by IRES and driven by one promoter can also be used.

The expression of a reporter gene is operably associated with an exogenous promoter. Useful promoters that may be used in the invention include any promoter known in the art that is suitable for the expression a marker gene in the corresponding organism. More specifically, a preferred promoter is any promoter active in pre-implantation embryos. Even more specifically, preferred promoters include, but are not limited to, a ubiquitin promoter, such as the human ubiquitin C promoter, the human ubiquitin 1 (hUb1) promoter, (see co-pending U.S. patent application Ser. No. 10/705,432), the SV40 early promoter region, the promoter contained in the 3′ long terminal repeat of Rous sarcoma virus, the regulatory sequences of the metallothionein gene, mouse or human cytomegalovirus IE promoter (Gossen et al. (1995) Proc. Nat. Acad. Sci. USA 89:5547-5551), PGK (phosphoglycerate kinase), Nanog (Chambers et al. (2003) Cell 113:643-655), ROSA26 (U.S. Patent Application Pub. No. 2003/0084468), and any other suitable promoter known by skilled artisans. According to a preferred embodiment, the promoter operably associated with the reporter gene is the human ubiquitin C promoter (hUbC).

The vector also comprises nucleic acid sequences that are homologous to a region within the Y chromosome gene and/or locus being targeted which guide the targeting vector to recombine at the target locus.

Homologous sequences can be generated through standard PCR methodology based on oligonucleotide primers specific for a suitable target locus. Once generated and amplified, homologous sequences can be ligated to the vector through standard techniques in a position and orientation appropriate for the desired recombination. In one specific embodiment of the invention, homology arms target a region on a mouse Y chromosome approximately 7 kb downstream from the 3′ UTR of the Sry gene (ENSEMBL accession number ENSMUSG00000043876, DNA sequence location AC140408.2.14022.19681). More specifically, mouse Y chromosome regions approximately 3518141 to 3523030 (according to ENSEMBL nomenclature) include a NheI restriction site (located at DNA sequence AC140408.2.31830.42342). The presence of the NheI restriction site in the homologous sequences is especially useful for the creation and use of an insertion vector, as it provides linearization site prior to transfection.

According to a preferred embodiment, the targeting vector also includes a selectable marker gene. The selectable marker gene facilitates the process of amplifying the plasmid and identifying ES cells that have been successfully transfected with the vector. The selectable marker is any marker suitable for serving these needs. Any selectable marker gene known in the art can be used, including a drug resistance gene, such as, for example, neomycin phosphotransferase (neo^{r}), hygromycin B phosphotransferase (hyg^{r}), puromycin-N-acetyltransferase (puro^{r}), blasticidin S deaminase (bsr^{r}), xanthine/guanine phosphoribosyl transferase (gtp), Herpes simplex virus thymidine kinase (HSV-tk) and fusions of tk with neo^{r}, hyg^{r} or puro^{r}. Suitable selection agents for drug resistance genes include G418 (with neo^{r}), puromycin (with puro^{r}), hygromycin B (with hyg^{r}), blasticidin S (with bsr^{r}), mycophenolic acid and 6-thioxanthine (with gtp) and gancyclovir or 1(2′-deoxy-2′-fluoro-beta-D-arabinofuranosyl)-5-iodouracil (FIAU) (with HSV-tk). Other selection agents include toxins such as, for example, diphtheria toxin A fragment (DTA). In a preferred embodiment, the selectable marker gene is the neo^{r} gene. The selectable marker gene is operably associated with at least one promoter. For example, the selectable marker gene may be operably associated with a first promoter (e.g., human ubiquitin C) that drives expression of the selectable marker gene in a first system (e.g., a eukaryotic system) and a second promoter (e.g., EM7) that drives expression of the selectable marker gene in a second system (e.g., a prokaryotic system). The ability to express the selectable marker gene in both a prokaryotic system and a eukaryotic system provides advantages for both the amplification process of the vector (usually performed in bacteria) and for the identifying eukaryotic cells that have been successfully transfected with the vector. Any of the promoters discussed above operably associated with the reporter gene may be used in connection with the selectable marker gene. In one embodiment, both a human ubiquitin C promoter and a prokaryotic EM7 promoter are associated with the selectable marker gene, which, in one embodiment, comprises the neo^{r} gene.

The selectable marker gene may be engineered as a conditional allele so that its presence and/or activity in transfected cells may be regulated and/or eliminated. Accordingly, the selectable marker gene may be positioned between (e.g., flanked by) a pair of site-specific recombination sites, such as loxP sites (recognized by Cre recombinase), Frt sites (recognized by Flp recombinase), or any other suitable recombination site/recombinase system known in the art. If the site-specific recombination sites are placed in the same orientation as defined by their asymmetric core region, the intervening sequences (i.e., the sequences located between the site-specific recombination sites) are excised after exposure to the appropriate recombinase. If the site-specific recombination sites are placed in the opposite orientation with respect to one another as defined by their asymmetric core region, the intervening sequences are inverted after exposure to the appropriate recombinase.

In an embodiment where a non-targeting vector is used which integrates a reporter gene randomly into the genome of the recipient cell, the non-targeting vector will include all the elements of the targeting vector except it will lack sequences homologous to a region of the Y chromosome.

Identification of Genetically Mutated and/or Modified Eukaryotic Cells

Methods for identifying genetically modified cells are well known in the art and include, for example, (a) Southern blotting; (b) long PCR; (c) quantitative PCR using TaqMan® (Lie and Petropoulos (1998) Curr Opin Biotechnol 9:43-8), molecular beacons (Tan et al. (2000) Chemistry, 6:1107-11) SYBR green, LUX primers (Invitrogen), and qzyme® (BD Bioscience); (d) fluorescence in situ hybridization (FISH) (Laan et al. (1995) Hum Genet 96:275-80) or comparative genomic hybridization (CGH) (Forozan et al. (1997) Trends Genet 13:405-9); (e) isothermal DNA amplification (Lizardi et al. (1998) Nat Genet 19:225-32); (f) quantitative hybridization to the immobilized target locus (Southern (1975) J. Mol. Biol. 98:503); (g) loss of polymorphic markers unique to the targeted locus; (h) observation of the products of the introduced exogenous gene; see also, U.S. Pat. No. 6,586,251, and Valenzuela et al. (2003) supra.

In one embodiment, a two-step procedure may be used to identify eukaryotic cells that have undergone Y chromosome tagging by the methods of the present invention. First, cells successfully transfected with a targeting vector comprising a selectable marker, e.g., neo^{r}, can be identified on the basis of their ability to survive in a culture medium containing an appropriate selection agent, such as G418 in the case of neo^{r}. Next, cells that have successfully undergone targeted homologous recombination (i.e., cells comprising desired Y chromosome modification) may be distinguished from ES cells that have undergone random integration by analysis for the presence of the marker gene at the desired Y chromosome locus by any conventional technique, such as, for example, PCR, Southern blotting, etc. See Joyner et al. (2000) The Practical Approach Series, 212, for example, for a discussion of techniques for confirming targeted recombinants.

Other features of the invention will become apparent in the course of the following descriptions of exemplary embodiments which are given for illustration of the invention and are not intended to be limiting thereof.

### EXAMPLES

The following examples are put forth so as to provide those of ordinary skill in the art with examples of how to make and use the methods, compositions and animals of the invention, and are not intended to limit the scope of the invention. Efforts have been made to ensure accuracy with respect to numbers used (e.g., amounts, temperature, etc.) but some experimental deviations are to be expected as is known to one of skill in the art. Unless indicated otherwise, parts are parts by weight, molecular weight is average molecular weight, temperature is in degrees Centigrade, and pressure is at or near atmospheric.

### Example 1

### Generation of a Transgenic Animal with a Tagged Y Chromosome

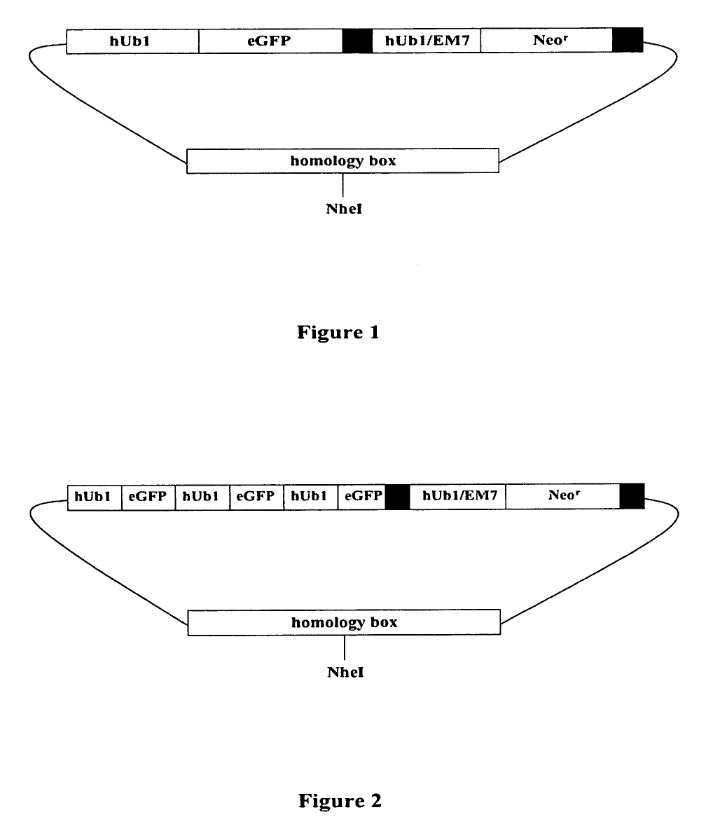

FIG. 1 is an example of an insertion targeting vector of the present invention. The vector includes a plasmid backbone, homologous sequences positioned within the backbone and, in the 5′ to 3′ direction, a human ubiquitin C promoter (hUbC), an eGFP gene operably associated with the hUbC promoter, a first Frt site, hUbC and prokaryotic EM7 promoters, a neomycin resistance gene operably associated with the hUbC and EM7 promoters, and a second Frt site.

Homologous sequences are generated by PCR amplification of a mouse Y chromosome locus approximately 7 kb downstream from the 3′ UTR of the Sry gene, approximately 3518141 to 3523030 (according to ENSEMBL nomenclature). A NheI restriction site is located at Y chromosome region approximately 3520465. The homologous sequences generated are approximately 2 kb long, but other sizes can be used.

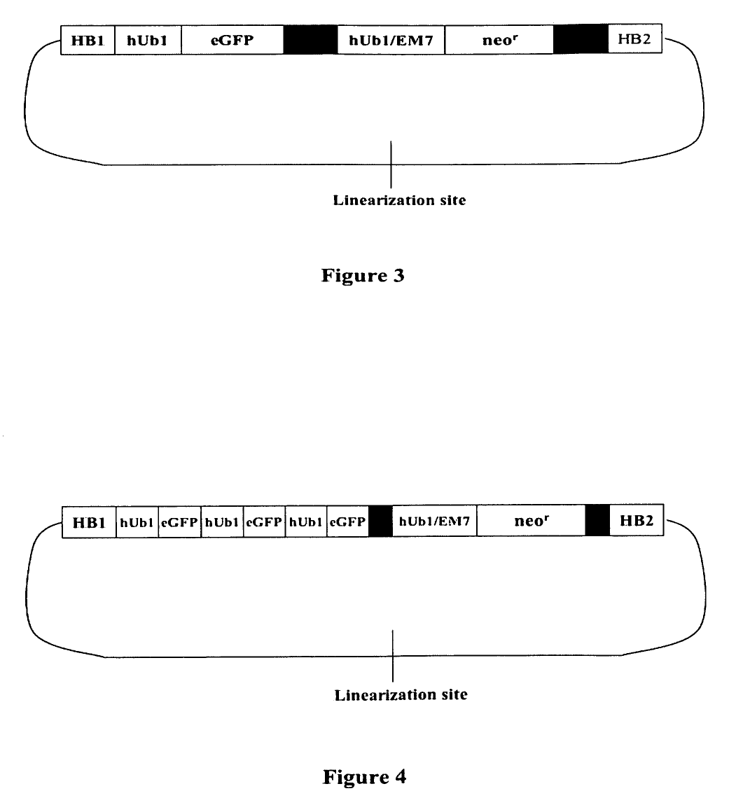

The final insertion vector is amplified in bacteria to a sufficient quantity for transfection into ES cells. The insertion vector is linearized at the NheI site within the homologous sequences prior to transfection and is transfected into cultured ES cells. The cells in which the insertion vector is introduced successfully can be selected by exposure to any number of selection agents, such as G418, or other suitable selection agent depending on the selectable marker gene present on the vector. Surviving cells are grown and tested to identify cells in which the insertion vector is successfully integrated into the desired Y chromosome. Alternatively, any other suitable vector, such as the vectors described herein, particularly the vectors illustrated in FIGS. 2-4, even more particularly the replacement vectors of FIGS. 3 and 4, can be used to modify the Y chromosome. Any such vector can be linearized prior to transfection.

ES cells with a tagged Y chromosome are microinjected into recipient embryos according to standard techniques. The recipient embryo is then implanted into a pseudopregnant female for gestation of the embryo and delivery of offspring. The offspring are then analyzed to identify offspring that have received the tagged Y chromosome and are capable of transmitting the tagged Y chromosome to future progeny, also though known techniques.

We claim:

1. A method of generating a eukaryotic cell having a detectable Y chromosome, comprising:
(a) generating a Y chromosome targeting vector, wherein the targeting vector comprises a 5′ homology arm, a promoter, a reporter gene operably associated with the promoter, and a 3′ homology arm, wherein the 5′ and 3′ homology arms are homologous to a region of the Y chromosome; and (b) introducing the targeting vector of step (a) into a eukaryotic cell.

2. The method of claim 1, wherein the reporter gene encodes a fluorescent protein.

3. The method of claim 2, wherein the fluorescent protein is selected from the group consisting of cyano fluorescent protein (CFP), green fluorescent protein (GFP), enhanced GFP (eGFP), yellow fluorescent protein (YFP), enhanced YFP (eYFP), blue fluorescent protein (BFP), enhanced BFP (eBFP), and a red fluorescent protein.

4. The method of claim 1, wherein the promoter is a promoter active in pre-implantation development.

5. The method of claim 4, wherein the promoter is one of a human ubiquitin C promoter, SV40 early promoter, Rous sarcoma virus promoter, human cytomegalovirus IE promoter, PGK promoter, and ROSA26 promoter.

6. The method of claim 1, wherein the eukaryotic cell is an embryonic stem (ES) cell.

7. The method of claim 6, wherein the ES cell is a mouse ES cell.

8. The method of claim 7, wherein the 5′ and 3′ homology arms are homologous to a region of the mouse Y chromosome approximately 7 kb downstream from the 3′ UTR of the Sty gene.

9. The method of claim 8, wherein the mouse Y chromosome region is approximately 3518141 to 3523030 (according to ENSEMBL nomenclature).

10. The method of claim 1, wherein the targeting vector is an insertion vector or a replacement vector.

11. The method of claim 1, wherein the targeting vector further comprises a selectable marker gene operably associated with a promoter.

12. The method of claim 11, wherein the selectable marker gene is a drug resistance gene.

13. The method of claim 12, wherein the drug resistance gene is selected from the group consisting of neomycin phosphotransferase (neo^{r}), hygromycin B phosphotransferase (hyg^{r}), puromycin-N-acetyltransferase (puro^{r}) and herpes simplex virus-tyrosine kinase (HSV-tk).

14. A method for generating a transgenic animal comprising a detectable Y chromosome, the method comprising:
(a) generating a Y chromosome targeting vector, wherein the targeting vector comprises a 5′ homology arm, a promoter, a reporter gene operably associated with the promoter, and a 3′ homology arm, wherein the 5′ and 3′ homology arms are homologous to a region of the Y chromosome;
(b) introducing the Y chromosome targeting vector of step (a) into a eukaryotic embryonic stem cell;
(c) introducing the cell of step (b) into an embryo; and
(d) introducing the embryo into a surrogate mother for gestation.

15. The method of claim 14, wherein the embryo is a pre-implantation embryo.

16. The method of claim 15, wherein the embryo is a blastocyst stage embryo.

17. A method of identifying a female embryo, comprising:
(a) generating a Y chromosome targeting vector, wherein the targeting vector comprises a 5′ homology arm, a promoter, a reporter gene operably associated with the eukaryotic promoter, and a 3′ homology arm, wherein the 5′ and 3′ homology arms are homologous to a region of the Y chromosome;
(b) introducing the Y chromosome targeting vector into an eukaryotic embryonic stem (ES) cell, wherein cells having a Y chromosome become detectable;
(c) introducing the cell into an embryo;
(d) introducing the embryo into a surrogate mother for gestation, wherein an animal is produced;
(e) identifying a male animal produced in step (d) producing sperm having a detectable Y chromosome;
(f) breeding the male animal of step (e) to a female animal such that embryos are generated;
(g) harvesting the embryo of step (f); and
(h) identifying an embryo lacking a detectable Y chromosome, wherein such an embryo is female.

18. A eukaryotic cell having a detectable Y chromosome generated by the method of claim 1.

19. A transgenic animal comprising a detectable Y chromosome generated by the method of claim 14.
